(19) United States
(12) Patent Application Publication
(10) Pub. No.: US 2026/0356789 A1
(43) Pub. Date: Nov. 12, 2026

(54) PROCESS FOR PREPARING ALKYL METHACRYLATES

(71) Applicants: DOW GLOBAL TECHNOLOGIES LLC, Midland, MI (US); ROHM AND HAAS COMPANY, Collegeville (PA)

(72) Inventors: Kirk Limbach, Dresher, PA (US); Reetam Chakrabarti, Phoenixville, PA (US); William G. Worley, Missouri City, TX (US)

(21) Appl. No.: 19/125,791

(22) PCT Filed: Nov. 20, 2023
(86) PCT No.: PCT/US2023/080436

§ 371 Date: Apr. 30, 2025

Related U.S. Application Data
(60) Provisional application No. 63/431,036, filed on Dec. 8, 2022.

(51) Int. Cl. C07C 67/39; C07C 45/75; C07C 45/82; C07C 47/22; C07C 69/54

(52) U.S. Cl. CPC C07C 67/39; C07C 45/75; C07C 45/82; C07C 47/22; C07C 69/54

(57) ABSTRACT

Provided is process preparing an alkyl methacrylate comprising: (a) reacting propionaldehyde and formaldehyde produce methacrolein intermediate stream; (b) subjecting methacrolein intermediate stream produced in step (a) to at least one phase separation and at least one distillation to reduce amount of methacrolein dimer and water in methacrolein intermediate stream, wherein amount of methacrolein dimer exiting at least one phase separation and at least one distillation is less than 10 weight % based on weight of methacrolein intermediate stream exiting at least one phase separation and at least one distillation, and amount of water exiting at least one phase separation and at least one distillation is less than 10 weight % based on total weight of methacrolein intermediate stream exiting at least one phase separation and at least one distillation; and (c) reacting the methacrolein with an alkyl alcohol in an oxidative esterification reaction system in the presence of at least one inhibitor, an oxygen-containing gas, and a catalyst comprising gold particles and particles of at least one metal oxide to produce product stream comprising an alkyl methacrylate. Product stream comprises from 0.1 to 5000 ppm of alkyl isobutyrate and from 0.01 to 5 weight % of at least one Michael addition product based on weight of product stream.

### FIELD OF THE INVENTION

This invention relates to a process for preparing alkyl methacrylates.

### BACKGROUND

The conversion of an aldehyde and alcohol in the presence of oxygen to a carboxylic ester via oxidative esterification, and in particular the conversion of methacrolein and methanol in the presence of oxygen to methyl methacrylate, has been known for many years. For example, U.S. Pat. No. 4,249,019 discloses the use of a palladium (Pd)-lead (Pb) catalyst and other catalysts for this purpose.

Typical process configurations have included slurry catalyst bubble column reactors and slurry catalyst continuous stirred tank reactors (CSTR). Slurry type reactors for this chemistry typically use a catalyst of less than 200 μm size, and U.S. Pat. No. 6,228,800 discloses the use of an egg-shell type catalyst of less than 200 μm size for slurry reactions. Issues with the use of slurry catalysts stem from catalyst attrition which may limit the life of the catalyst and make filtration of the product stream difficult. According to CN1931824, these problems can be addressed through the use of a larger size catalyst charged to a fixed bed reactor. However, as noted in U.S. Patent Application Publication No. 2016/0251301, the use of larger catalyst particles leads to a reduced space-time yield and other potential disadvantages.

Fixed bed technology with larger catalyst particles has been implemented in U.S. Pat. No. 4,520,125, which discloses the use of a 4 mm diameter catalyst in a fixed bed system. The reactor feed in that case was relatively dilute, as it is in more recent discussions of fixed bed technology for this chemistry such as U.S. Patent Application Publication No. 2016/0251301 and U.S. Patent Application Publication No. 2016/0280628.

In commercial production facilities, the oxidative esterification reactors are followed by a separation section consisting of distillation columns to purify the product and recycle dewatered and otherwise purified unreacted reactants (see, e.g., U.S. Pat. No. 5,969,178) where the product and recycle often constitute the majority of the product stream. In part, this is because methanol is typically provided to the oxidative esterification reactor in excess to maximize the conversion of valuable methacrolein (see, e.g., U.S. Pat. No. 7,326,806).

Feed concentration of methacrolein into the oxidative esterification reactor varies in the literature from very low (see, e.g., U.S. Pat. No. 5,892,102) to around 35 wt % (see, e.g., U.S. Pat. No. 8,461,373). Methanol is typically the major constituent of the feed and the recycle stream that returns to the oxidative esterification reactor from the downstream separations section.

Catalysts for this chemistry have included various noble metals such as palladium-based catalysts including palladium-lead catalyst (see, e.g., U.S. Pat. No. 4,249,019) and gold-based or gold-containing catalysts (see, e.g., U.S. Pat. Nos. 7,326,806 and 8,461,373).

It is desirable to maximize selectivity and reduce the formation of all byproducts. In particular, byproduct methyl isobutyrate (MIB) is critical to reduce because it is difficult to separate from the product MMA and is undesirable in the product.

### STATEMENT OF INVENTION

One aspect of the invention provides a process for preparing an alkyl methacrylate comprising:
(a) reacting propionaldehyde and formaldehyde to produce a methacrolein intermediate stream;
(b) subjecting the methacrolein intermediate stream produced in step (a) to at least one phase separation and at least one distillation to reduce an amount of methacrolein dimer and water in the methacrolein intermediate stream, wherein the amount of methacrolein dimer exiting the at least one phase separation and at least one distillation is less than 10 weight % based on the total weight of the methacrolein intermediate stream exiting the at least one phase separation and the at least one distillation, and the amount of water exiting the at least one phase separation and the at least one distillation is less than 10 weight % based on the total weight of the methacrolein intermediate stream exiting the at least one phase separation and the at least one distillation; and
(c) reacting the methacrolein with an alkyl alcohol in an oxidative esterification reaction system in the presence of at least one inhibitor, an oxygen-containing gas, and a catalyst comprising gold particles and particles of at least one metal oxide to produce a product stream comprising an alkyl methacrylate, wherein the metal of the at least one metal oxide is selected from aluminum, titanium, lanthanides, zirconium, alkali metals, alkaline earth metals, nickel, cobalt, zinc, magnesium, tellurium, antimony, rhenium, tungsten, and bismuth;
wherein the product stream comprises from 0.1 to 5000 ppm of an alkyl isobutyrate and from 0.01 to 5 weight % of at least one Michael addition product based on the total weight of the product stream.

### DETAILED DESCRIPTION

All percentage compositions are weight percentages (wt %), all amounts provided in terms of parts per million (ppm) are on the basis of weight, and all temperatures are in °C, unless otherwise indicated. Averages are arithmetic averages unless otherwise indicated. An “average concentration” is the arithmetic average of the concentration entering a region and the concentration exiting the region, where the region is an individual reactor, a reactor system, or a zone within a reactor or reactor system. An “average ratio” is the ratio of the average concentration of one component relative to the average concentration of another component. For example, the average ratio of alcohol to methacrolein in a reactor system is calculated by dividing the average concentration of alcohol entering and exiting the reactor system by the average concentration of methacrolein entering and exiting the reactor system.

A noble metal is any of gold, platinum, iridium, osmium, silver, palladium, rhodium and ruthenium. More than one noble metal may be present in the catalyst, in which case the limits apply to the total of all noble metals.

The “catalyst center” is the centroid of the catalyst particle, i.e., the mean position of all points in all coordinate directions. A diameter is any linear dimension passing through the catalyst center and the average diameter is the arithmetic mean of all possible diameters. The aspect ratio is the ratio of the longest to the shortest diameters.

A reactor system refers to one or more reactors where a designated reaction takes place. For example, the oxidative esterification of methacrolein to produce an alkyl methacrylate may be the designated reaction that takes place in the reactor system. The reactor system may comprise a single reactor or a plurality of reactors. Additionally, the reactor system may be subdivided into multiple zones, i.e., a multizone reactor system. Zones may be defined by physical separation, such as by walls or barriers that define separate areas, or by differences in the reaction conditions, such as, for example, pressure, temperature, composition or concentration of the catalyst, reactants, or other reaction components such as inert materials, pH modifiers, etc. For example, the reactor system may comprise a single reactor comprising a single zone, a single reactor comprising multiple zones, multiple reactors comprising a single zone in each reactor, multiple reactors where one or more reactors has a single zone and one or more reactors that comprise multiple zones, or multiple reactors each comprising multiple zones. By definition, a reactor system comprising multiple reactors would be considered a multizone reactor system. An example of a multizone reactor may be a continuous tubular reactor comprising multiple zones, including one or more mixing zones, a cooling zone, and one or more catalyst zones where the reaction takes place. Another example of a multizone single reactor may be a stirred bed reactor comprising internal walls containing the catalyst that defines a catalyst zone through which liquid reactants are circulated, and a feed/removal zone outside of the catalyst zone where the reactants enter the reactor and products exit the reactor. When referring to the average concentration or any ratio of the reactor system, the average concentration or ratio is calculated based on what enters the reactor system and what exits the reactor system.

One aspect of the invention provides a process for preparing an alkyl methacrylate comprising reacting propionaldehyde and formaldehyde to produce a methacrolein intermediate stream, subjecting the methacrolein intermediate stream to at least one phase separation and at least one distillation, and reacting the methacrolein with an alkyl alcohol in an oxidative esterification reaction system to produce an alkyl methacrylate.

To produce methacrolein from propionaldehyde and formaldehyde, a catalyst stream is provided by mixing water and an amine-acid catalyst. The water and catalyst may be mixed in a catalyst tank prior to entering the reactor. The amine-acid catalyst is capable of catalyzing the Mannich condensation of propionaldehyde and formaldehyde to methacrolein. The Mannich condensation process is known in the art, for example, as described in U.S. Pat. No. 4,496,770 and U.S. Pat. No. 7,141,702. Suitable amine-acid catalysts include, for example, those comprising a secondary amine and an acid.

Examples of suitable acids of the amine-acid catalysts include inorganic acids and organic mono-, di-, or polycarboxylic acids. Suitable carboxylic acids include, but are not limited to, aliphatic C₁-C₁₀ monocarboxylic acids, C₂-C₁₀ dicarboxylic acids, C₂-C₁₀ polycarboxylic acids. Preferred carboxylic acids comprise at least one of acetic acid, propionic acid, methoxyacetic acid, n-butyric acid, isobutyric acid, oxalic acid, succinic acid, tartaric acid, glutaric acid, adipic acid, maleic acid, fumaric acid, and combinations thereof. Preferred inorganic acids include sulfuric acid and phosphoric acid.

Suitable amines of the amine-acid catalysts include, for example, those of the formula NHR²R³, where R² and R³ are each independently C₁-C₁₀ alkyl, which are optionally substituted with an ether, hydroxyl, secondary amino or tertiary amino group, or R² and R³, together with the adjacent nitrogen, may form a C₅-C₇ heterocyclic ring, optionally containing a further nitrogen atom and/or an oxygen atom, and which are optionally substituted by a C₁-C₄ alkyl or C₁-C₄ hydroxyalkyl. Preferred amines comprise at least one of dimethylamine, diethylamine, methylethylamine, methylpropylamine, dipropylamine, dibutylamine, diisopropylamine, diisobutylamine, methylisopropylamine, methylisobutylamine, methyl-sec.-butylamine, methyl-(2-methylpentyl)-amine, methyl-(2-ethylhexyl)-amine, pyrrolidine, piperidine, morpholine, N-methylpiperazine, N-hydroxyethylpiperazine, piperazine, hexamethyleneimine, diethanolamine, methylethanolamine, methylcyclohexylamine, methylcyclopentylamine, and dicyclohexylamine, and combinations thereof.

Preferably, the amine-acid catalyst comprises dimethylamine and acetic acid. The molar ratio of the amine to acid may be such that the resulting pH is from 2.5 to 7. For example, the amine-acid catalyst may contain a molar ratio of dimethylamine to acetic acid in an amount of from 10:1 to 1:10, preferably of from 5:1 to 1:5, and more preferably of from 1:1 to 1.2:1.

The Mannich condensation reaction may be carried out by sending the catalyst stream and a reaction stream containing propionaldehyde, formaldehyde, and methanol to a reactor to produce a first intermediate stream containing methacrolein, methanol, and water via the Mannich condensation reaction. The reaction can be carried out under any suitable conditions at which the reaction proceeds. For example, the reaction can be conducted at a temperature of at least 20 °C and at least atmospheric pressure. Preferably, the reaction is conducted in the liquid phase at above 150 °C, e.g., 150-220 °C, and at superatmospheric pressure, i.e., greater than 1 bar. Preferably, the reaction is conducted in the liquid phase at a pressure ranging from greater than 1 bar to 150 bar, more preferably from 10 bar to 120 bar.

The molar ratio of propionaldehyde to formaldehyde is not particularly limited. For example, the reaction stream may contain a ratio of propionaldehyde to formaldehyde in an amount of from 1.1:1 to 1:2, preferably of from 1.1:1 to 1:1.5, and more preferably of from 1.05:1 to 1:1.05. The first intermediate stream is considered a “wet” methacrolein stream in that it comprises significant amounts of water, e.g., greater than 10 weight %, greater than 20 weight %, or more, based on the total weight of the first intermediate stream.

The propionaldehyde used to prepare the methacrolein can be prepared by the hydroformylation of ethylene. The hydroformylation process is known in the art, and is disclosed, for example, in U.S. Pat. Nos. 4,427,486, 5,087,763, 4,716,250, 4,731,486, and 5,288,916. The hydroformylation of ethylene to propionaldehyde comprises contacting ethylene with carbon monoxide and hydrogen in the presence of a hydroformylation catalyst. Examples of hydroformylation catalysts include, for example, metal-organophosphorus ligand complexes, such as organophosphines, organophosphites, and organophosphoramidites. The ratio of carbon monoxide to hydrogen may range from 1:10 to 100:1, preferably from 1:10 to 10:1. The hydroformylation process may be conducted at a temperature ranging from −25 °C to 200 °C, preferably from 50 °C to 120 °C.

Ethylene used to prepare propionaldehyde may be prepared from the dehydration of ethanol. For example, ethylene can be prepared by the acid-catalyzed dehydration of ethanol. Ethanol dehydration is known in the art and is disclosed, for example, in U.S. Pat. No. 9,249,066. Preferably, ethanol is sourced from renewable resources, such as plant materials or biomass, as opposed to ethanol prepared from petroleum based sources. For example, using bio-resourced ethanol alone in the process for producing MMA can result in up to 40% of the carbon atoms of the MMA (i.e., 2 of the 5 carbon atoms in the MMA) coming from renewable resources.

To further increase the renewable carbon content in the alkyl methacrylate, additional starting materials can also be prepared from renewable resources. For example, formaldehyde can be prepared from syngas, where the syngas can be prepared from biomass. Carbon monoxide, which can also be used in the preparation of propionaldehyde, can also be prepared from renewable resources, as disclosed by Li et al., ACS Nano, 2020, 14, 4, 4905-4915. Using these additional bio-resourced can further increase the amount of renewable carbon.

Alternatively, starting materials to produce the alkyl methacrylate can be prepared from recycled materials. For example, recycled carbon dioxide can be used to produce methanol, and the methanol can be used to produce formaldehyde.

Preferably, at least 40% of the carbon atoms in the alkyl methacrylate are derived from renewable or recycled content, more preferably at least 60%, even more preferably at least 80%, and still more preferably 100%.

The methanol and formaldehyde present in the reaction stream may be provided in the form of formalin. The formalin utilized in the process of the invention may comprise a saturated water solution containing formaldehyde in an amount of about 37 weight %, and methanol in an amount of from 10 to 15 weight %, based on the total weight of the formalin. The methanol present in the formalin can be advantageously used in the subsequent oxidative esterification process, which converts methacrolein in the presence of an alkyl alcohol to alkyl methacrylate, e.g., converting methacrolein in the presence of methanol to methyl methacrylate. In certain embodiments, methanol can be introduced at various locations in the process.

The first intermediate stream is subjected to at least one distillation step and to at least one phase separation step.

For example, in one embodiment, the first intermediate stream can be subjected to a phase separation to produce an organic phase, comprising water, methanol, and primarily methacrolein, and an aqueous phase, which comprises methacrolein, methanol, amine-acid catalyst, and primarily water. In this embodiment, the methacrolein may be present in the organic phase in an amount of at least 70 weight %, preferably at least 85 weight %, and more preferably at least 90 weight %, based on the total weight of the organic phase. The methanol may be present in the organic phase in an amount of less than 10 weight %, preferably less than 3 weight %, and more preferably less than 2.5 weight %, based on the total weight of the organic phase. While not wishing to be bound by theory, it is believed that operating the phase separator at low temperatures results in the organic phase containing lower amounts of methanol, which is beneficial for the downstream distillation of the organic phase. The phase separator may be operated at a temperature of less than 15 °C, preferably less than 10 °C, and more preferably less than 5 °C. The aqueous phase may comprise water in an amount of at least 70 weight %, preferably at least 75 weight %, and more preferably at least 80 weight %.

The organic phase can be distilled in a first distillation column to produce a second intermediate stream and an overhead stream. The first distillation column may be operated as a stripping column, wherein the overheads vapors are condensed without any liquid being refluxed back to the column. The ratio of the second intermediate stream exiting the first distillation column to the organic phase entering the first distillation column may range from 1:10 to 8:10, preferably from 3:10 to 7:10, and more preferably from 5:10 to 6:10. The second intermediate stream contains water, methanol, and primarily methacrolein. Water may be present in the second intermediate stream in an amount of less than 2 weight %, preferably less than 1 weight %, and more preferably less than 0.5 weight %, based on the total weight of the second intermediate stream. Methacrolein may be present in the second intermediate stream in an amount of at least 70 weight %, preferably 85 weight %, and more preferably 95 weight %, based on the total weight of the second intermediate stream. The overhead stream contains water, methanol, and primarily methacrolein. Water may be present in the overhead stream in an amount of greater than 2 weight %, preferably greater than 3 weight %, and more preferably greater than 4 weight %. Preferably, at least part of the overhead stream is recycled to the phase separator.

The second intermediate stream may then be distilled in a second distillation column to produce a first product stream and a waste stream. The first product stream contains water, methanol, and primarily methacrolein. The methacrolein may be present in the first product stream in an amount of at least 70 weight %, preferably at least 85 weight %, and more preferably at least 95 weight %, based on the total weight of the first product stream. In embodiments when the product of the subsequent oxidative esterification reaction is methyl methacrylate, methanol may be present in the first product stream in an amount of less than 30 weight %, preferably less than 10 weight %, and more preferably less than 2 weight %, based on the total weight of the first product stream. The methacrolein and methanol may be present in the first product stream in a combined amount of at least 97 weight %, preferably at least 98 weight %, and more preferably at least 99 weight %. Water may be present in the first product stream in an amount of less than 2 weight %, preferably less than 1 weight %, and more preferably less than 0.5 weight %, based on the total weight of the first product stream. In embodiments where the methacrolein is used to form an alkyl methacrylate other than methyl methacrylate in the subsequent oxidative esterification reaction, the methanol is preferably substantially removed from the product stream to avoid the formation of methyl methacrylate. The waste stream contains undesired organic compounds from the process, e.g., methacrolein dimer, 2-methyl-2-pentenal, inhibitor, and other heavy organic compounds from the process.

The aqueous phase may be distilled in a third distillation column to produce a second product stream, a bottoms stream, and a side draw stream. The second product stream contains water, methanol, and methacrolein. Water may be present in the second product stream in an amount of less than 5 weight %, preferably less than 2 weight %, and even more preferably less than 1 weight %, based on the total weight of the second product stream. Methacrolein may be present in the second product stream in an amount of greater than 25 weight %, preferably greater than 35 weight %, and more preferably greater than 45 weight %, based on the total weight of the second product stream. In embodiments where methyl methacrylate is the product of the subsequent oxidative esterification reaction, methanol may be present in the second product stream in an amount of greater than 25 weight %, preferably greater than 40 weight %, and more preferably greater than 55 weight %, based on the total weight of the second product stream. In embodiments where an alkyl methacrylate is the desired product of the subsequent oxidative esterification reaction, the methanol is substantially removed from the second product stream. The bottoms stream contains amine-acid catalyst of the catalyst stream that is recovered. Preferably at least part of the bottoms stream is recycled to the catalyst stream, which in preferred embodiments is mixed in the catalyst tank. The side draw stream may contain primarily water and certain organic compounds from the process. The side draw stream may contain methanol in an amount of less than 2 weight %, preferably less than 1.5 weight %, and more preferably less than 1 weight %.

In an alternative embodiment, the first intermediate stream can be subjected to at least one distillation step prior to the at least one phase separation step. In this embodiment, the first intermediate stream is distilled in a first distillation column to produce a second intermediate stream comprising water, methanol, residual formaldehyde and propionaldehyde, and primarily methacrolein. Methacrolein is preferably present in the second intermediate stream in an amount of at least 70 weight %, preferably at least 80 weight %, and more preferably at least 85 weight % of the total weight of the second intermediate stream. The bottoms stream of the first distillation column comprises methacrolein, amine-acid catalyst, and primarily water. Methacrolein may be present in the bottoms stream of the first distillation column in an amount less than 10 weight %, preferably less than 7.5 weight %, and more preferably less than 5 weight % of the total weight of the bottoms stream. The bottoms stream of the first distillation column may be recycled to the first distillation column to recover additional methacrolein. The amine-acid catalyst in the bottoms stream may be recovered in second distillation column and recycled to the catalyst tank or discarded.

The second intermediate stream can then be subjected to a phase separation to produce an organic phase, comprising water, methanol, and primarily methacrolein, and an aqueous phase comprising methacrolein, methanol, and primarily water. Methacrolein may be present in the organic phase in an amount of at least 75 weight %, preferably at least 85 weight %, and more preferably at least 95 weight % based on the total weight of the organic phase. Water may be present in the organic phase in an amount less than 8 weight %, preferably less than 5 weight %, more preferably less than 2 weight %, and even more preferably less than 1 weight % based on the total weight of the organic phase. The aqueous phase contains methacrolein, methanol, and primarily water. The organic phase may be subjected to further distillation in a third distillation column to further remove unwanted byproducts to produce a product stream comprising primarily methacrolein. In embodiments where an alkyl methacrylate other than methyl methacrylate is the desired product of the subsequent oxidative esterification reaction, the methanol may be removed from the product stream prior to entering the oxidative esterification reactor.

Inhibitors can be introduced into the process through one or more locations, for example, the catalyst tank, the reactor, the phase separator, any of the distillation columns, and in the intermediate or product streams. Suitable inhibitors include, for example, 4-hydroxy-2,2,6,6-tetramethylpiperidin-1-oxyl (4-Hydroxy-TEMPO).

The propionaldehyde in the reaction stream may be prepared by the hydroformylation of ethylene. The hydroformylation process is known in the art, for example, as described in U.S. Pat. Nos. 4,427,486, 5,087,763, 4,716,250, 4,731,486, and 5,288,916. The hydroformylation of ethylene to propionaldehyde involves contacting ethylene with CO and hydrogen in the presence of a hydroformylation catalyst. Suitable hydroformylation catalysts include, for example, metal-organophosphorous ligand complexes. Suitable organophosphorous ligands include, for example, organophosphines, organophosphites, and organophosphoramidites. In certain embodiments, the ratio of CO to hydrogen is in the range of from 1:10 to 100:1, preferably of from 1:10 to 10:1. In certain embodiments, the hydroformylation reaction is conducted at a reaction temperature of from −25 °C to 200 °C, preferably of from 50 °C to 120 °C.

Any of the product streams comprising methacrolein may be utilized in a downstream oxidative esterification (“OER”) process to form an alkyl methacrylate from the methacrolein and an alkyl alcohol in an OER reactor system. Preferably, the product stream entering the OER reactor system comprises less than 10 weight % methacrolein dimer, more preferably less than 6 weight % methacrolein dimer, even more preferably less than 2 weight % methacrolein dimer, and still more preferably less than 1 weight % methacrolein dimer, and less than 10 weight % water, more preferably less than 8 weight % water, still more preferably less than 6 weight % water, and even more preferably less than 5 weight % water, based on the total weight of the stream entering the OER reactor system.

The OER reactor system may comprise a single reactor or a plurality of reactors. Additionally, the reactor system may be subdivided into multiple zones, i.e., a multizone reactor system. Zones may be defined by physical separation, such as by walls or barriers that define separate areas, or by differences in the reaction conditions, such as, for example, pressure, temperature, composition or concentration of the catalyst, reactants, or other reaction components such as inert materials, pH modifiers, etc. For example, the reactor system may comprise a single reactor comprising a single zone, a single reactor comprising multiple zones, multiple reactors comprising a single zone in each reactor, multiple reactors where one or more reactors has a single zone and one or more reactors that comprise multiple zones, or multiple reactors each comprising multiple zones. By definition, a reactor system comprising multiple reactors would be considered a multizone reactor system. An example of a multizone reactor may be a continuous tubular reactor comprising multiple zones, including one or more mixing zones, a cooling zone, and one or more catalyst zones where the reaction takes place. Another example of a multizone single reactor may be a stirred bed reactor comprising internal walls containing the catalyst that defines a catalyst zone through which liquid reactants are circulated, and a feed/removal zone outside of the catalyst zone where the reactants enter the reactor and products exit the reactor. When referring to the average concentration or any ratio of the reactor system, the average concentration or ratio is calculated based on what enters the reactor system and what exits the reactor system.

The reactor system may comprise a reactor configured as a fluidized bed reactor, a fixed bed reactor, a trickle bed reactor, a packed bubble column reactor, or a stirred bed reactor. Preferably, the reactor system comprises a packed bubble column reactor.

The catalyst may be present in the form of a slurry or a fixed bed depending on the reactor in which the catalyst is present. For example, a slurry catalyst can be used in a stirred bed reactor or a fluidized bed reactor, whereas a fixed bed catalyst can be used in a fixed bed reactor, trickle bed reactor, or a packed bubble column reactor. Preferably, the reactor is in the form of a fixed bed reactor.

The size of the catalyst can be selected based on the type of reactor. For example, a slurry catalyst may have an average particle diameter less than 200 μm, such as, for example, from 10 μm to 200 μm. A fixed bed catalyst may have an average particle diameter 200 μm or greater, such as, for example, from 200 μm to 30 mm. Preferably, the average diameter of the catalyst particle is at least 200 μm, more preferably at least 400 μm, even more preferably at least 600 μm, and still more preferably at least 800 μm; preferably no more than 30 mm, more preferably no more than 20 mm, and even more preferably no more than 10 mm.

The catalyst preferably comprises gold particles and particles of at least one metal oxide. Preferably, the metal of the at least one metal oxide is selected from aluminum, titanium, lanthanides, zirconium, alkali metals, alkaline earth metals, nickel, cobalt, zinc, magnesium, tellurium, antimony, rhenium, tungsten, and bismuth. Preferably, the metal of the at least one metal oxide is selected from nickel and titanium.

The gold particles preferably have an average diameter of less than 12 nm, preferably less than 10 nm, and more preferably less than 8 nm. The standard deviation of the average diameter of the gold particles is +/−4 nm, preferably +/−2.5 nm, and more preferably +/−2 nm. As used herein, the standard deviation is calculated by the following equation:

$$standard deviation = \sqrt{\frac{{\sum }_{i = 1}^{n} \vert x - \overline{x} \vert}{n}}$$

where x is the size of each particle, x is the mean of the n number of particles, and n is at least 500.

The particles of at least one metal oxide preferably have an average diameter of less than 5 times the average diameter of the gold particles, more preferably an average diameter of less than 4 times the average diameter of the gold particles, even more preferably an average particle diameter of less than 3 times the average diameter of the gold particles, still more preferably an average particle diameter of less than 2 times the average diameter of the gold particles, and yet more preferably an average particle diameter of less than 1.5 times the average diameter of the gold particles. Preferably, the particles of at least one metal oxide have an average diameter at least the half the average diameter of the gold particles, and more preferably at least the same as the average diameter of the gold particles

The amount by weight of the gold particles with respect to the amount of the particles of at least one metal oxide may range from 1:1 to 1:20. Preferably, the weight ratio of gold particles to particles of at least one metal oxide ranges from 1:2 to 1:15, more preferably from 1:3 to 1:10, even more preferably from 1:4 to 1:9, and still more preferably from 1:5 to 1:8.

Preferably, the gold particles are evenly distributed among the particles of at least one metal oxide. As used herein, the term “evenly distributed” means the gold particles are randomly dispersed among the particles of at least one metal oxide with substantially no agglomeration of the gold particles. Preferably, at least 80% of the total number of the gold particles are present in a particle having an average diameter less than 12 nm. More preferably, at least 90% of the total number of the gold particles are present in a particle having an average diameter less than 12 nm. Even more preferably, at least 95% of the total number of gold particles are present in a particle having an average diameter less than 12 nm.

Preferably, at least 75% of the gold particles by number of gold particles are within at least 20 nm of a metal oxide particle. As used herein, the phrase “within at least X nm” means that an edge of a gold particle is within X nm of an edge of the metal oxide particle nearest the gold particle. Preferably, at least 75% of the gold particles are within at least 15 nm of a metal oxide particle, more preferably within at least 12 nm of a metal oxide particle, and even more preferably within at least 10 nm of a metal oxide particle.

More preferably, at least 75% of the gold particles by number of the gold particles are within at least 20 nm of two metal oxide particles, i.e., an edge of the gold particle is within at least 20 nm of an edge of the two metal oxide particles nearest the gold particle. Preferably, at least 75% of the gold particles are within at least 15 nm of two metal oxide particles, more preferably within at least 12 nm of two metal oxide particles, and even more preferably within at least 10 nm of two metal oxide particles.

Even more preferably, at least 75% of the gold particles by number of the gold particles are within at least 20 nm of at least three metal oxide particles, i.e., an edge of the gold particle is within at least 20 nm of an edge of at least the three metal oxide particles nearest the gold particle. Preferably, at least 75% of the gold particles are within at least 15 nm of at least three metal oxide particles, more preferably within at least 12 nm of at least three metal oxide nanoparticles, and even more preferably within at least 10 nm of at least three metal oxide particles.

The gold particles in the catalyst may be disposed on a surface of a support material. Preferably, the support material is a particle of an oxide material; preferably γ-, δ-, or θ-alumina, silica, magnesia, titania, zirconia, hafnia, vanadia, niobium oxide, tantalum oxide, ceria, yttria, lanthanum oxide or a combination thereof. Preferably, in portions of the catalyst comprising the noble metal, the support has a surface area greater than 10 m²/g, preferably greater than 30 m²/g, preferably greater than 50 m²/g, preferably greater than 100 m²/g, preferably greater than 120 m²/g. In portions of the catalyst which comprise little or no noble metal, the support may have a surface area less than 50 m²/g, preferably less than 20 m²/g. The average diameter of the support and the average diameter of the final catalyst particle are not significantly different.

Preferably, the aspect ratio of the catalyst particle is no more than 10:1, preferably no more than 5:1, preferably no more than 3:1, preferably no more than 2:1, preferably no more than 1.5:1, preferably no more than 1.1:1. Preferred shapes for the catalyst particle include spheres, cylinders, rectangular solids, rings, multi-lobed shapes (e.g., cloverleaf cross section), shapes having multiple holes and “wagon wheels;” preferably spheres. Irregular shapes may also be used.

The gold particles can be dispersed throughout the catalyst or have varying concentration densities, such as, for example, a gradient concentration or layered structure. Preferably, at least 90 wt % of the gold particles are in the outer 70% of catalyst volume (i.e., the volume of an average catalyst particle), preferably the outer 60% of catalyst volume, preferably the outer 50%, preferably the outer 40%, preferably the outer 35%, preferably in the outer 30%, preferably in the outer 25%. Preferably, the outer volume of any particle shape is calculated for a volume having a constant distance from its inner surface to its outer surface (the surface of the particle), measured along a line perpendicular to the outer surface. For example, for a spherical particle the outer x % of volume is a spherical shell whose outer surface is the surface of the particle and whose volume is x % of the volume of the entire sphere. Preferably, at least 95 wt % of the noble metal is in the outer volume of the catalyst, preferably at least 97 wt %, preferably at least 99 wt %. Preferably, at least 90 wt % (preferably at least 95 wt %, preferably at least 97 wt %, preferably at least 99 wt %) of the noble metal(s) is within a distance from the surface that is no more than 30% of the catalyst diameter, preferably no more than 25%, preferably no more than 20%, preferably no more than 15%, preferably no more than 10%, preferably no more than 8%. Distance from the surface is measured along a line which is perpendicular to the surface.

Preferably, the catalyst comprises gold particles and particles of at least one metal oxide on a support material comprising silica. Preferably, the gold particles and particles of at least one metal oxide form an eggshell structure on the support particles. The eggshell layer may have a thickness of 500 microns or less, preferably 250 microns or less, and more preferably 100 microns or less.

Preferably, at least 0.1% by weight of the total weight of the gold particles are exposed on a surface of the catalyst, where the surface includes both the outer surface and pores of the catalyst. As used herein, the term “exposed” means that at least a portion of the gold particle is not covered by another gold particle or a particle of at least one metal oxide, i.e., the reactants can directly contact the gold particle. The gold particles may therefore be disposed within a pore of the support material and still be exposed by virtue of the reactant being able to directly contact the gold particle within the pore. More preferably, at least 0.25% by weight of the total weight of the gold particles are exposed on the surface of the catalyst, even more preferably, at least 0.5% by weight of the total weight of the gold particles are exposed on the surface of the catalyst, and still more preferably, at least 1% by weight of the total weight of the gold particles are exposed on the surface of the catalyst.

The catalyst may be prepared by a process that comprises providing a support and providing particles of at least one oxide of a metal on a surface of the support, contacting the support with a gold salt, and heating the support at a temperature ranging from 50 °C to 600 °C for a time ranging from at least 0.1 h to 48 h to convert the gold salt to gold nanoparticles having an average diameter less than 12 nm and a standard deviation of +/−4 nm, wherein at least 75% by number of the gold nanoparticles are positioned within 20 nm of a particle of the oxide of the metal.

The particles of at least one metal oxide may be formed on the support by first contacting the support with a salt of the metal and then oxidizing the metal in an environment comprising an oxygen-containing gas, e.g., air or oxygen.

To form gold nanoparticles on the support, the support is contacted with a gold salt, preferably in the form of an aqueous solution comprising the gold salt. The gold salt is then converted to particles of metallic gold by heating the support at a temperature ranging from 50 °C to 600 °C for a time ranging from 0.1 h to 48 h.

In one embodiment, the step of heating the support to convert the gold salt to metallic gold may be conducted in the presence of an oxygen-containing gas, such as air or oxygen, to calcine the catalyst. Calcination may be conducted at a temperature ranging from 150 °C to 500 °C, preferably from 200 °C to 450 °C. Calcination is a preferred process for converting the gold salt to metallic gold because it can simultaneously form the particles of a metal oxide from metal salts.

In an alternative embodiment, the step of heating the support to convert the gold salt to metallic gold may be conducted in the presence of a reducing gas comprising at least 0.1% by volume of a reducing agent.

In another alternative embodiment, the step of heating the support to convert the gold salt to metallic gold may be conducted in the presence of an inert atmosphere, in which case the inert gas allows the gold salt to self-reduce to metallic gold.

In yet another alternative embodiment, the step of heating the support to convert the gold salt to metallic gold may be conducted in the liquid phase in the presence of a solvent and a reductant, wherein the ratio of reductant to solvent is at least 0.01. In this embodiment, the support is heated at a temperature ranging from 50 °C to less than the boiling point of the solvent.

Contacting support with a gold salt may be performed by several different methods. For example, contacting the support with a gold salt may be performed by impregnating the support with an aqueous solution comprising the gold salt, dip coating the support with a solution comprising the gold salt, spray coating the support with a solution comprising the gold salt, or sequentially impregnating the support by impregnating the support with a first solution to fill at least 80% by volume of any pores that may be present in the support, followed by impregnating the support with a second solution comprising the gold salt. When contacting the support with a solution or aqueous solution of a gold salt, the support may first be dried before the support is heated to convert the gold salt to metallic gold particles.

Preferably, the catalyst is produced by precipitating the gold and metal (i.e., the metal of the metal oxide) from an aqueous solution of metal salts in the presence of the support. In one preferred embodiment, the catalyst is produced by contacting an aqueous solution of a suitable gold precursor salt and nickel salt with a porous inorganic oxide such that the pores are filled with the solution and the water is then removed by drying. The resulting material is then converted into a finished catalyst by calcination or reduction, to decompose the gold salts and metal salts into gold and metal oxides. Preferably, a C₂-C₁₈ thiol comprising at least one hydroxyl or carboxylic acid substituent is present in the solution. Preferably, the C₂-C₁₈ thiol comprising at least one hydroxyl or carboxylic acid substituent has from 2 to 12 carbon atoms, preferably 2 to 8, preferably 3 to 6. Preferably, the thiol compound comprises no more than 4 total hydroxyl and carboxylic acid groups, preferably no more than 3, preferably no more than 2. Preferably, the thiol compound has no more than 2 thiol groups, preferably no more than one. If the thiol compound comprises carboxylic acid substituents, they may be present in the acid form, conjugate base form or a mixture thereof. Especially preferred thiol compounds include thiomalic acid, 3-mercaptopropionic acid, thioglycolic acid, 2-mercaptoethanol and 1-thioglycerol, including their conjugate bases.

The catalyst bed may further comprise inert or acidic materials. Preferred inert or acidic materials include, e.g., alumina, clay, glass, silica carbide and quartz. Preferably, the inert or acidic materials located before and/or after the catalyst bed have an average diameter equal to or greater than that of the catalyst, preferably 1 to 30 mm; preferably at least 2 mm; preferably no greater than 30 mm, preferably no greater than 10 mm, preferably no greater than 7 mm.

In the OER reactor system, an alkyl methacrylate is produced by reacting methacrolein with an alkyl alcohol in the presence of an oxygen-containing gas. The alkyl group of the alkyl methacrylate is a straight or branched C₁ to C₁₂ alkyl group. The alkyl alcohol comprises a straight or branched alcohol comprising from 1 to 12 carbon atoms. Preferably, the alkyl alcohol is selected from the group consisting of methanol, ethanol, propanol, butanol, hexanol, 2-ethylhexanol, and octanol, in all of their isomeric forms. More preferably, the alkyl alcohol is selected from the group consisting of methanol, ethanol, butanol, and 2-ethylhexanol. Even more preferably, the alkyl alcohol is methanol.

The catalyst bed, which may comprise a slurry bed or fixed bed, comprises the catalyst particles. The OER reactor produces a product stream comprising a liquid phase comprising methacrolein, the alkyl alcohol and the alkyl methacrylate and a gaseous phase comprising oxygen. The liquid phase may further comprise byproducts, e.g., Michael addition products, methacrolein dialkyl acetals, such as, for example, methacrolein dimethyl acetal (MDA) or methacrolein dibutyl acetal, and an isobutyrate of an alkyl alcohol, such as, for example, methyl isobutyrate (MIB) or butyl isobutyrate (BIB). Without taking steps to control its formation, alkyl isobutyrates may be present in an alkyl methacrylate product stream in amounts in excess of 1 wt % (10,000 ppm) relative to the total weight of alkyl methacrylate, methacrolein and alkyl alcohol in the product stream exiting the OER system. Alkyl isobutyrates can be difficult to separate from the alkyl methacrylate. Therefore, the present invention seeks to limit the amount of alkyl isobutyrates that are formed such that the amount of alkyl isobutyrate in the product stream ranges from 0.1 ppm to 5000 ppm, preferably from 0.1 to 4000 ppm, more preferably from 0.1 to 3000 ppm, even more preferably from 0.1 to 2500 ppm, still more preferably from 0.1 to 2000 ppm, and yet more preferably from 0.1 to 1000 ppm, based on the total weight of the product stream. Preferably, the amount of Michael products in the product stream is ranges from 0.01 to 5 weight %, more preferably from 0.01 to 3 weight %, still more preferably from 0.01 to 2 weight %, and even more preferably from 0.01 to 1 weight %, based on the total weight of the product stream. Preferably, the amount of acetals and hemiacetals of methacrolein in the product stream ranges from 0.01 to 10 weight %, more preferably from 0.01 to 5 weight %, and even more preferably from 0.01 to 3 weight %, based on the total weight of the alkyl methacrylate and acetals and hemiacetals of methacrolein in the product stream exiting the OER system.

Preferably, the concentration of alkyl alcohol entering the OER system is greater than 32 wt % based on the total weight of alkyl alcohol and methacrolein entering the reactor system. More preferably, the concentration of alkyl alcohol entering the OER system is greater than 35 wt %, and even more preferably greater than 40 wt % based on the total weight of alkyl alcohol and methacrolein entering the reactor system. Preferably, the concentration of alkyl alcohol entering the OER system is less than 75 wt % based on the total weight of alkyl alcohol and methacrolein entering the reactor system. More preferably, the concentration of alkyl alcohol entering the OER system is less than 60 wt %, and even more preferably less than 50 wt % based on the total weight of alkyl alcohol and methacrolein entering the reactor system.

Preferably, the concentration of alkyl alcohol in the liquid phase product stream exiting the OER system ranges from 15 wt % to 95 wt % based on the total weight of the liquid phase product stream exiting the OER system. For example, the concentration of alkyl alcohol in the liquid phase product stream exiting the OER system may be at least 20 wt %, at least 25 wt %, or at least 30 wt % based on the total weight of the liquid phase product stream exiting the OER system. Preferably, the concentration of alkyl alcohol in the liquid phase product stream exiting the OER system is less than 90 wt %, more preferably less than 80 wt %, even more preferably less than 70 wt %, still more preferably less than 60 wt %, and yet more preferably less than 50 wt % based on the total weight of the liquid phase product stream exiting the OER system.

Preferably, the average concentration of alkyl alcohol in the OER system (i.e., the arithmetic average of the concentration of the alkyl alcohol entering and exiting the OER system) is greater than 70 wt % based on the average total weight of alkyl alcohol and methacrolein entering the reactor system (i.e., the arithmetic average of the total weight of methanol and methacrolein entering the OER system and the total weight of methanol and methacrolein exiting the OER system). More preferably, the average concentration of alkyl alcohol in the OER system is greater than 75 wt % based on the average total weight of alkyl alcohol and methacrolein entering and exiting the reactor system.

It is preferred that the average weight ratio of alkyl alcohol to methacrolein in the OER system ranges from 20:1 to 2:1, where the average weight ratio is based on the average concentration of alkyl alcohol entering and exiting the OER system and the average concentration of methacrolein entering and exiting the OER system.

One example of an OER system comprises a multizone or multi-reactor system. In a first zone or reactor, the average concentration of alkyl alcohol in the first zone or reactor ranges from 50 wt % to 80 wt % based on the average total amount of alkyl alcohol and methacrolein entering and exiting the first zone or reactor. The final zone or reactor has an average alkyl alcohol concentration ranging from 80 wt % to 100 wt % based on the average total amount of alkyl alcohol and methacrolein entering and exiting the final zone or reactor. Between the first zone or reactor and the final zone or reactor, the reactor mixture may be cooled and/or additional oxygen may be added, such as, for example, by adding air to a gas phase entering the final zone or reactor.

Preferably, oxygen concentration in a gas stream exiting the OER system is at least 1 mol %, more preferably at least 2 mol %, even more preferably at least 2.5 mol %, still more preferably at least 3 mol %, yet more preferably at least 3.5 mol %, even yet more preferably at least 4 mol %, and most preferably at least 4.5 mol %, based on the total volume of the gas stream exiting the OER system. Preferably, the oxygen concentration in a gas stream exiting the OER system is no more than 7.5 mol %, preferably no more than 7.25 mol %, preferably no more than 7 mol %, based on the total amount of the gas stream exiting the OER system.

Preferably, the liquid phase in the OER system is at a temperature from 40 to 120 °C; preferably at least 50 °C, and preferably at least 55 °C. The temperature of the liquid phase in the OER system is preferably no more than 110 °C, and preferably no more than 100 °C. When the OER system comprises more than one reactor and/or more than one zone, the temperature in each reactor and/or zone may be the same or different. For example, a reaction mixture exiting a reactor or zone may be cooled prior to entering the next reactor or zone.

Preferably, the catalyst bed in the OER system is at a pressure from 1 to 150 bar (100 to 15000 kPa). Without wishing to be limited by theory, it is believed that operating the OER system at increased pressure will lower the amount of MIB present in the product stream by increasing the amount of oxygen present in the liquid phase. Therefore, the pressure in the catalyst bed of the OER system may be at least 10 bar, more preferably at least 20 bar, even and more preferably at least 30 bar, and preferably less than 150 bar, and more preferably less than 120 bar. When the OER system comprises more than one reactor and/or zone, the pressure in each reactor and/or zone may be the same or different.

The heterogeneous noble metal-containing catalyst in the OER system may be present in an amount ranging from 0.02 kg to 2 kg of catalyst for every gram-mole of alkyl methacrylate exiting the reactor system over the course of 1 hour. Preferably, the heterogeneous noble metal-containing catalyst in the OER system is present in an amount of at least 0.02 kg to 0.5 kg of catalyst, for every gram-mole of alkyl methacrylate exiting the reactor system over the course of 1 hour. Preferably, the heterogeneous noble metal-containing catalyst in the OER system is present in an amount of less than 0.4 kg of catalyst, more preferably less than 0.3 kg of catalyst, still more preferably less than 0.25 kg of catalyst, and even more preferably less than 0.2 kg of catalyst for every gram-mole of alkyl methacrylate exiting the reactor system over the course of 1 hour.

The amount of alkyl methacrylate exiting the reactor is dependent on the conversion of methacrolein in the OER system. For example, at 50% conversion of methacrolein entering the OER system, 2 moles of methacrolein would be required for every mole of alkyl methacrylate produced. In this example, the heterogeneous noble metal-containing catalyst in the OER system may be present in an amount ranging from 0.01 to 1 kg of catalyst for every gram-mole of methacrolein entering the reactor system over the course of 1 hour. At 25% conversion of methacrolein entering the OER system, 4 moles of methacrolein would be required for every mole of alkyl methacrylate produced, and the heterogeneous noble metal-containing catalyst in the OER system may be present in an amount ranging from 0.005 to 0.5 kg of catalyst for every gram-mole of methacrolein entering the reactor system over the course of 1 hour. At 75% conversion of methacrolein entering the OER system, 1.33 moles of methacrolein would be required for every mole of alkyl methacrylate produced, and the heterogeneous noble metal-containing catalyst in the OER system may be present in an amount ranging from 0.015 to 1.5 kg of catalyst for every gram-mole of methacrolein entering the reactor system over the course of 1 hour. Disregarding any external recycle streams, the OER system preferably exhibits at least 25% conversion of methacrolein to alkyl methacrylate, more preferably at least 35% conversion, and even more preferably at least 40% conversion of methacrolein to alkyl methacrylate in the OER system. Addition of an external recycle stream that recycles unreacted methacrolein to the OER system can also be used to improve the overall conversion efficiency of the process.

When the noble metal-containing catalyst comprises gold, the gold may be present in an amount ranging from 0.0001 kg to 0.1 kg for every gram-mole of alkyl methacrylate exiting the reactor system over the course of 1 hour. Preferably, the gold is present in an amount of at least 0.0001 kg to 0.005 kg for every gram-mole of alkyl methacrylate exiting the reactor system over the course of 1 hour. Preferably, the gold is present in an amount less than 0.004 kg for every gram-mole of alkyl methacrylate exiting the reactor system over the course of 1 hour.

In terms of the amount of heterogeneous noble metal-containing catalyst in the OER system with respect to the amount of methacrolein entering the reactor system, at 50% conversion of methacrolein entering the OER system, the gold in the heterogeneous noble metal-containing catalyst in the OER system may be present in an amount ranging from 0.00005 to 0.05 kg of gold for every gram-mole of methacrolein entering the reactor system over the course of 1 hour. At 25% conversion of methacrolein entering the OER system, the gold in the heterogeneous noble metal-containing catalyst in the OER system may be present in an amount ranging from 0.000025 to 0.025 kg of catalyst for every gram-mole of methacrolein entering the reactor system over the course of 1 hour. At 75% conversion of methacrolein entering the OER system, the gold in the heterogeneous noble metal-containing catalyst in the OER system may be present in an amount ranging from 0.000075 to 0.075 kg of catalyst for every gram-mole of methacrolein entering the reactor system over the course of 1 hour.

The pH in the catalyst bed may range from 2 to 10. Some catalysts may be deactivated in acidic conditions. Therefore, when the catalyst is not acid resistant, the pH in the catalyst bed is from 4 to 10; preferably at least 5, preferably at least 5.5; preferably no greater than 9, preferably no greater than 8, preferably no greater than 7.5.

To increase the pH in the reactor system, a base material may be added. The base material may comprise an Arrhenius base (i.e., a compound that dissociates in water to form hydroxide ions), a Lewis base (i.e., a compound capable of donating a pair of electrons), or a Bronsted-Lowry base (i.e., a compound capable of accepting a proton). Examples of Arrhenius bases include, but are not limited to, hydroxides of alkali and alkali earth metals. Examples of Lewis bases include, but are not limited to, amines, sulfates, and phosphines. Examples of Bronsted-Lowry bases include, but are not limited to, halides, nitrates, nitrites, chlorites, chlorates, etc. Ammonia can be either a Lewis base or a Bronsted-Lowry base.

The present inventors have discovered that high localized concentrations of base materials in the reactor system can cause the formation of unwanted Michael adduct as byproducts. Therefore, to aid in the minimization of the formation of Michael adducts, the base material is preferably mixed with at least one other material prior to entering the reactor system. Preferably, the base material is introduced at a position external to the reactor system and mixed with one or more reactants or diluents to form a base-containing stream. For example, the base material may be mixed with the alkyl alcohol, water, or a non-reactive solvent, i.e., a solvent that does not negatively impact the formation of the alkyl methacrylate in the reactor system. The position external to the reactor system may be a mixing vessel. Alternatively, the position external to the reactor may be a line through which components travel to the reactor system, such as a feed line or a recycle line, in which sufficient mixing occurs, such as by turbulent flow, baffles, jet mixer, or other mixing method.

Preferably, the amount of the base material in the base-containing stream is 50 wt % or less based on the total weight of the base-containing stream, preferably 25 wt % or less, preferably 20 wt % or less, preferably 15 wt % or less, preferably 10 wt % or less, preferably 5 wt % or less, or preferably 1 wt % or less. The base material is preferably diluted by a factor of less than 1:2, such as, less than 1:3, less than 1:4, less than 1:5, less than 1:10, less than 1:20, or less than 1:100, relative to the total weight of the base-containing stream prior to entering the reactor system. Preferably, the amount of base material added to the OER reactor system is less than 10 weight %, more preferably less than 5 weight %, and even more preferably less than 2 weight %, based on the total weight of reactants in the OER reactor system.

Preferably, the base-containing stream is sufficiently mixed to avoid localized spikes in the concentration of the base material within the base-containing stream before it is added to the reactor system. For example, it is preferred that the base-containing stream reach at least 95% degree of homogeneity, i.e., variations in the concentration of the base material deviate within +/−5% of the average concentration of base material for the base-containing stream prior to entering the reactor system. Preferably, the base-containing stream reaches 95% degree of homogeneity within 4 minutes of introduction of the base material, more preferably within 2 minutes, and even more preferably within 1 minute of introduction of the base material.

For a mixing vessel, the time required for an additive to reach a 95% degree of homogeneity is defined at Θ₉₅, which can be calculated by the method disclosed by Grenville and Nienow, The Handbook of Industrial Mixing, Pages 507-509, which gives the following expression for a stirred tank in turbulent flow:

$${θ}_{95} = 5.2 \frac{{T}^{1.5} {H}^{0.5}}{{D}^{2} {N}_{P}^{1 / 3} N}$$

where T is the tank diameter, H is the liquid height, D is the impeller diameter, Np is the characteristic power number of the impeller(s), and N is the impeller speed. Similar expressions exist for static mixers, jet mixed vessels, etc.

Preferably, no base material is added to the reactor system, either internal or external to the reactor system. Preferably, when no base material is added to the reactor system, the noble metal-containing catalyst comprises an acid-resistant catalyst such as a catalyst comprised of gold and titanium-containing particles. Operating the OER system in the absence of a base material may provide several advantages. One advantage is the increased selectivity and space time yield (STY) due to lower production of Michael adducts. Another advantage is the reduction in cost due to the reduced cost to treat aqueous waste. Aqueous waste exiting an oxidative esterification process in which a base material was used can produce large quantities of inorganic salts, which can be difficult or impossible to treat with biological water treatment processes. This in turn, may require the use of other waste treatment process, such as incineration.

The product stream from the OER system is preferably subjected to at least one distillation and at least one phase separation to purify and recover components within the product stream. For example, the product stream contains unreacted methacrolein and alkyl alcohol that can be separated and returned to the OER system. Acetals and hemiacetals of methacrolein are preferably subjected to a hydrolysis reaction to recover additional methacrolein and alkyl alcohol. Michael addition products and alkyl isobutyrates present in the product stream are preferably removed.

Preferably, the product stream is fed to an alcohol recovery distillation column which provides an overhead stream rich in alkyl alcohol and methacrolein; preferably this stream is recycled back to the OER system. Some hydrolysis of the acetals and hemiacetals of methacrolein may occur in the alcohol recovery distillation column, allowing the recovery of additional alkyl alcohol and methacrolein in the alcohol recovery distillation column.

The bottoms stream from the alkyl alcohol recovery distillation column comprises the alkyl methacrylate, an isobutyrate of the alkyl alcohol, methacrylic acid, salts and water. The bottoms stream further comprises acetals and hemiacetals of methacrolein that were not hydrolyzed in the alcohol recovery distillation column. In one embodiment, the bottoms stream from the alkyl alcohol recovery distillation column is sent to an acetal hydrolysis reactor for additional hydrolysis of the acetals and hemiacetals of methacrolein followed by phase separation to separate the organic phase from the aqueous phase. In an alternative embodiment, the acetals and hemiacetals of methacrolein may be hydrolyzed in a separate acetal hydrolysis reactor following a phase separation of the alkyl alcohol recovery bottoms stream. It may be necessary to add water to the organic phase to ensure that there is sufficient water for the methacrolein dialkyl acetal hydrolysis; these amounts may be determined from the composition of the organic phase. An acid stream may also be added to the hydrolysis reactor to ensure adequate methacrolein dialkyl acetal removal. Preferably, the amount of acetals and hemiacetals of methacrolein exiting the acetal hydrolysis reactor and the phase separator ranges from 0.01 to 100 ppm, more preferably from 0.01 to 25 ppm, and even more preferably from 0.01 to 5 ppm based on the total weight of the stream exiting the acetal hydrolysis reactor and the phase separator.

In either embodiment, the organic phase that has been subjected to hydrolysis in the acetal reactor is then sent to a heavies removal column to remove Michael addition products. Preferably, the overhead stream of the heavies removal column comprises 0.01 to 1 weight %, more preferably from 0.01 to 0.5 weight %, and even more preferably from 0.01 to 0.25 weight % of Michael addition products based on the total weight of the overhead stream of the heavies removal column.

The overhead stream of the heavies removal column is then sent to an alkyl isobutyrate removal column to further reduce the amount of the alkyl isobutyrate in the product stream. Preferably, the amount of alkyl isobutyrate in the bottoms stream exiting the alkyl isobutyrate column ranges from 0.01 to 800 ppm, more preferably from 0.01 to 600 ppm, and even more preferably from 0.01 to 400 ppm based on the total weight of the bottoms stream exiting the alkyl isobutyrate column.

The bottoms stream of the alkyl isobutyrate column may be sent to an alkyl methacrylate product column to further purify the alkyl methacrylate. For example, process inhibitors which may have been added during any of the distillation or phase separation processes, may be removed and recycled.

What is claimed is:

1. A process for preparing an alkyl methacrylate comprising:
(a) reacting propionaldehyde and formaldehyde to produce a methacrolein intermediate stream;
(b) subjecting the methacrolein intermediate stream produced in step (a) to at least one phase separation and at least one distillation to reduce an amount of methacrolein dimer and water in the methacrolein intermediate stream, wherein the amount of methacrolein dimer exiting the at least one phase separation and at least one distillation is less than 10 weight % based on the total weight of the methacrolein intermediate stream exiting the at least one phase separation and the at least one distillation, and the amount of water exiting the at least one phase separation and the at least one distillation is less than 10 weight % based on the total weight of the methacrolein intermediate stream exiting the at least one phase separation and the at least one distillation;
(c) reacting the methacrolein with an alkyl alcohol in an oxidative esterification reaction system in the presence of at least one inhibitor, an oxygen-containing gas, and a catalyst comprising gold particles and particles of at least one metal oxide to produce a product stream comprising an alkyl methacrylate, wherein the metal of the at least one metal oxide is selected from aluminum, titanium, lanthanides, zirconium, alkali metals, alkaline earth metals, nickel, cobalt, zinc, magnesium, tellurium, antimony, rhenium, tungsten, and bismuth;
wherein the product stream comprises from 0.1 to 5000 ppm of an alkyl isobutyrate and from 0.01 to 5 weight % of at least one Michael addition product based on the total weight of the product stream.

2. The process of claim 1, wherein the propionaldehyde is produced by contacting ethylene with CO and H₂ in the presence of a hydroformylation catalyst.

3. The process of any one of the preceding claims, wherein the alkyl alcohol is a straight or branched alcohol comprising from 1 to 8 carbon atoms.

4. The process of claim 3, wherein the alkyl alcohol is selected from the group consisting of methanol, ethanol, propanol, butanol, hexanol, 2-ethylhexanol, and octanol, in all of their isomeric forms.

5. The process of any one of the preceding claims, methacrolein dimer exiting the at least one phase separation and at least one distillation is less than 5 weight % based on the total weight of the methacrolein intermediate stream exiting the at least one phase separation and the at least one distillation, and the amount of water exiting the at least one phase separation and the at least one distillation is less than 5 weight % based on the total weight of the methacrolein intermediate stream exiting the at least one phase separation and the at least one distillation.

6. The process of any one of the preceding claims, further comprising adding a base material to the oxidative esterification reactor system in an amount less than 10 weight % based on the total weight of reactants in the oxidative esterification reaction system.

7. The process of any one of the preceding claims, wherein the product stream comprises 0.1 to 1000 ppm of an alkyl isobutyrate based on the total weight of the product stream.

8. The process of any one of the preceding claims, wherein the product stream comprises from 0.01 to 1 weight % of at least one Michael addition product based on the total weight of the product stream.

9. The process of any one of the preceding claims, wherein the gold particles have an average diameter of less than 12 nm.

10. The process of any one of the preceding claims, wherein the metal of the at least one metal oxide is nickel or titanium.

11. The process of any one of the preceding claims, wherein the catalyst has an average particle diameter ranging from 200 μm to 30 mm.

12. The process of any one of the preceding claims, wherein the particles of at least one metal oxide preferably have an average diameter of less than 5 times the average diameter of the gold particles.

13. The process of any one of the preceding claims, wherein at least 75% of the gold particle are within at least 20 nm of a particle of at least one metal oxide.
